(19) United States
(12) Patent Application Publication
(10) Pub. No.: US 2007/0079826 A1
(43) Pub. Date: Apr. 12, 2007

(54) Apparatus for delivering humidified gases

(75) Inventors: Martin Paul Friedrich Kramer, Auckland (NZ); Ian Douglas Makinson, Auckland (NZ); Philip James Biggs, Auckland (NZ); Philip John Dickinson, Auckland (NZ)

Correspondence Address: TREXLER, BUSHNELL, GIANGIORGI,;BLACKSTONE & MARR, LTD., 105 WEST ADAMS STREET, SUITE 3600, CHICAGO, IL, 60603, US

(73) Assignee: Fisher & Paykel Healthcare Limited, Auckland (NZ)

(21) Appl. No.: 10/526,698

(22) PCT Filed: Sep. 17, 2003
(86) PCT No.: PCT/NZ03/00214

§ 371 Date: Sep. 12, 2005

(30) Foreign Application Priority Data
Sep. 17, 2002 (NZ) 521446
Aug. 20, 2003 (NZ) 527734

(51) Int. Cl. A61M 11/00

(52) U.S. Cl. 128/200.14

(57) ABSTRACT

An apparatus for delivering humidified gases has a connection manifold (8) adapted to connect with inlet (5) and outlet (6) ports of a slide-on water chamber (2) in a single slide on motion. Connection of the gases inlet (5) and gases outlet (6) ports as well as any additional electrical and/or pneumatic connections are all made in the same slide on motion. The water chamber (2) may include inwardly extending elongate extension tubes (30,31) and at least one of the extension tubes may also have an air bleed aperture (33) to aid filling of the chamber (2).

### BACKGROUND TO THE INVENTION

i) Field of the Invention

The present invention relates to apparatus for delivering humidified gases. In particular it relates to a humidifier arrangement for an integrated device providing respiratory assistance to patients, for example in consumer CPAP delivery devices.

ii) Summary of the Prior Art

Humidification systems are known which include a heater base and a disposable humidifier chamber which is fitted onto the heater base and within which a supply of water can be heated by the heater base. Air enters the humidifier chamber through an inlet air port in the roof of the chamber where it is humidified by the evaporation of water from the water supply before leaving the chamber through an exit port in the roof of the humidifier chamber.

Humidifier chambers of this type are also now used in compact and portable ventilation machines, for example machines intended for the home treatment of obstructive sleep apnoea (CPAP machines). Where the humidifier base is adapted for use with slide-on humidifier chambers, and the connection of the chamber to the machine is accomplished with a single sliding movement, the inlet air port is provided horizontally through the side of the chamber. Air enters the humidifier chamber through the inlet air port and the humidified air leaves the humidifier chamber into a breathing conduit through an exit port in the top of the humidifier chamber.

A disadvantage of these configurations is the need to disconnect the patient breathing conduit from the top of the humidifying chamber in a separate operation before removal of the chamber for the purpose of refilling. A further disadvantage of these configurations is that separate electrical wiring connections are required to make use of a heated respiratory conduit.

The present invention is described with particular reference to a CPAP delivery product. However it will be appreciated that the invention is applicable to any compact integrated humidified gases delivery product incorporating a pressurised gases supply and a humidification module. For example, physically similar devices may be used for patient ventilation, humidified oxygen delivery, and humidified insufflation.

### SUMMARY OF THE INVENTION

It is an object of the present invention to provide an apparatus for delivering humidified gases which at least goes some way towards overcoming the above disadvantages or which will at least provide the public with a useful choice.

In a first aspect the invention consists in an apparatus for use in humidified gases delivery treatment comprising:

a housing,

a pressurised gases supply within said housing,

a pressurised gases outlet in said housing in fluid connection with said pressurised gases supply and adapted to make fluid connection with an inlet of a humidifier in order to provide gases flow to a said humidifier,

a humidified gases return in said housing, adapted to make fluid connection with an outlet of a said humidifier in order to receive humidified gases from said humidifier,

a patient outlet in said housing, in fluid connection with said humidified gases return in order to receive humidified gases from said humidified gases return and provide humidified gases to said patient outlet, said patient outlet being in fluid connection with or adapted to make fluid connection with a breathing conduit for delivery of humidified gases to a patient.

Preferably said humidifier is a heatable water chamber, and said apparatus includes,

a chamber heating means connected to said housing and, said housing includes a humidifier engagement locating a said humidifier adjacent said chamber heating means, said chamber heating means adapted to vaporise liquid water in said water chamber in order to provide water vapour to said gases flow passing through said water chamber.

Preferably said humidification chamber has a base, and said chamber is engagable with said humidifier engagement via a single motion, and said single motion of engagement urges the base of said humidification chamber adjacent and in contact with said chamber heating means and makes a first fluid connection between said pressurised gases outlet and said humidifier inlet, and makes a second fluid connection between said humidified gases return and said humidifier outlet, with said first and second fluid connections being made in the direction of said single motion.

Preferably said pressurised gases outlet and said inlet of a said humidifier have between them first complementary male and female connectors, having a preferred insertion direction for completing a fluid connection by engagement of the male and female connectors,

said humidified gases return and said outlet of said humidifier have between them second complementary male and female connectors, having a preferred insertion direction for completing a fluid connection by engagement of the male and female connectors, said preferred insertion direction of said first connectors being the same as said preferred insertion direction of said second connectors, and being the same as at least the direction of a terminal part of said single motion.

Preferably said inlet of said humidifier and said outlet of said humidifier are each a female port,

and said pressurised gases outlet and said humidified gases return are each a resilient tubular projection fitting within respective female ports with said chamber engaged.

Preferably said protruding tubes of said pressurised gases outlet and humidified gases return have substantially parallel axis of extension, said chamber heating means includes a substantially planar heating plate, and said axis of extension of said tubes are at least substantially parallel with the plane of said heating plate.

Preferably said patient outlet includes a connector for receiving a breathing hose and at least one auxiliary electrical connection plug or socket or pneumatic connection plug or port, for a simultaneous connection when connecting a breathing circuit having complementary electrical or pneumatic connectors.

In a further aspect the invention consists in an apparatus for use in humidified gases delivery treatment comprising:

a container, with a surrounding wall and top, and an open bottom,

a heat conductive base enclosing said open bottom of said container,

a gases inlet to said container,

a gases outlet to said container,

a first elongate flow tube extending into said humidifier container from the inner periphery of said gases inlet, with an opening at a distal end of said flow tube being spaced from said wall of said chamber,

a second elongate flow tube extending into said humidifier container from the inner periphery of said gases outlet, with an opening at a distal and of said flow tube being spaced from said wall of said chamber,

said first and said second flow tubes being substantially parallel to each other, and substantially parallel to said base of said chamber, and

said gases inlet and said gases outlet facing the same direction, a preferred insertion direction, and

said preferred insertion direction is substantially parallel to the said base of said chamber, such that

said humidifier chamber may make operable engagement with a heater base in a single motion,

and fluid connections with said gases outlet and said gases inlet, being also made in said single motion.

Preferably said opening of said first flow tube faces a direction transverse to an axis of said first flow tube, and said opening of said second flow tube faces a direction transverse to an axis of said second flow tube.

Preferably said transverse direction is not downwards.

Preferably said transverse direct is upwards.

Preferably said chamber further includes a baffle between said first flow tube and said second flow tube.

Preferably said baffle extends from the roof of said chamber and terminates below the surface of water in said chamber when said chamber is filled to a maximum intended water level for use.

Preferably said second flow tube includes an air bleed orifice, said air bleed orifice being located in the top of said second elongate flow tube, and located toward the end of the elongate flow tube adjacent said gases outlet.

Preferably said gases inlet and said gases outlet of said humidifier chamber are

each a female port, and

said humidifier chamber is generally cylindrical, and said female ports open out to the cylindrical surface adjacent the top of the cylindrical wall.

To those skilled in the art to which the invention relates, many changes in construction and widely differing embodiments and applications of the invention will suggest themselves without departing from the scope of the invention as defined in the appended claims. The disclosures and the descriptions herein are purely illustrative and are not intended to be in any sense limiting.

### BRIEF DESCRIPTION OF THE DRAWINGS

Embodiments of the present invention will now be described with reference to the drawings.

FIG. 1 is a perspective view of a water chamber and CPAP machine according to an embodiment of the present invention showing the water chamber 2 separated from the CPAP machine 1.

FIG. 2 is a perspective view of the water chamber and CPAP machine of FIG. 1, showing the water chamber 2 engaged with the CPAP machine 1.

FIG. 3 is a perspective view of a CPAP machine and water chamber according to an alternative embodiment of the present invention.

FIG. 4 is a perspective view of a water chamber of the present invention showing hidden detail of the inlet and outlet extension tubes.

FIG. 5 is a sectioned side view of the water chamber of FIG. 4 sectioned through a mid-line of the outlet extension tube with the intended water level shown hatched.

FIG. 6 is a sectioned side view of the water chamber of FIG. 4, sectioned through a mid-line of the chamber with the water level of the chamber when tilted shown hatched.

FIG. 7 is a perspective view of an inlet/outlet extension tube according to an embodiment of the present invention showing snap-fit protrusions and locating/locking means.

FIG. 8 is a front view of a water chamber of the present invention showing the flanges and notches which co-operate with the extension tubes detailed in FIG. 7.

FIG. 9 is a perspective view of an outlet extension tube according to an embodiment of the present invention showing an air bleed slot.

FIG. 10 is a perspective view of a water chamber according to a further embodiment showing hidden detail of the inlet and outlet extension tubes.

### DETAILED DESCRIPTION

Embodiments of the present invention will now be described in more detail.

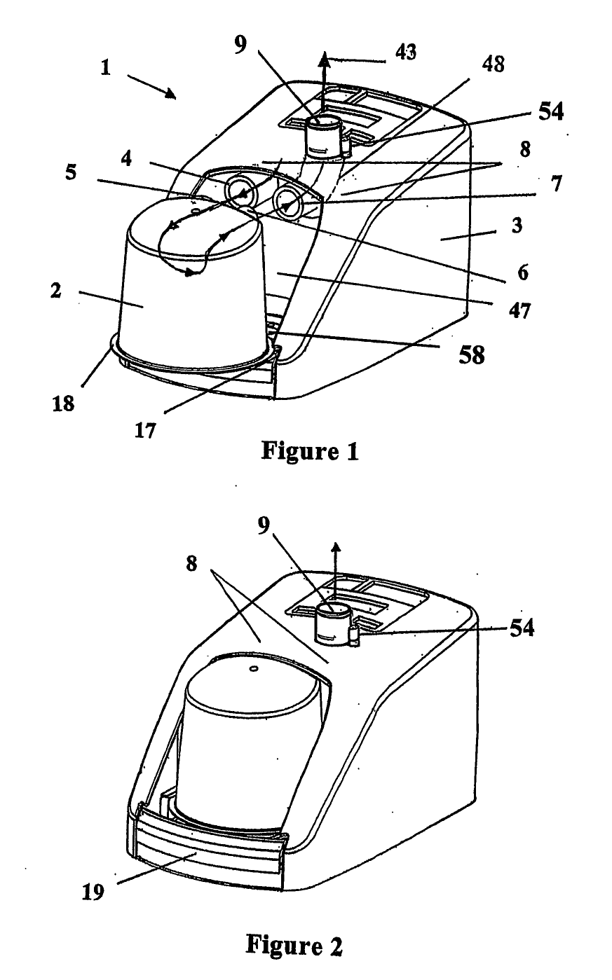

Referring to FIGS. 1 and 2, a preferred embodiment of the invention, in a CPAP machine has a housing containing a blower and a heater base, and a corresponding water chamber. A water chamber having a gases inlet port 5 and gases outlet port 6 is shown with a portable CPAP machine. The CPAP machine is adapted to receive slide-on humidifier chambers. The CPAP machine connects to the gases inlet/outlet ports of the water chamber through a connection manifold. Connection of the gases inlet and gases outlet ports are made to the connection manifold 8 of the CPAP machine in a single slide-on motion. The connection manifold 8 also provides an auxiliary outlet connection port 9 suitable for receiving a flexible respiratory conduit to deliver humidified air to a patient.

The CPAP machine includes a heater base 58 in a chamber receiving bay 47 to heat the water chamber. A securing arrangement is provided for locating and engaging the water chamber to the CPAP machine. The securing arrangement has a securing latch 19 and a slot 17 around the periphery of the chamber receiving bay 47. The slot co-operates with a flange 18 around the base of the water chamber to secure the chamber when in use. The securing latch 19 operates to prevent removal of the chamber once it has been engaged. The securing means and connection manifold are arranged with a parallel axis of operation, such that connection of the chamber inlet and outlet ports 5 & 6, to the connection manifold 8 is achieved together with the securing of the chamber into the CPAP machine in the same single slide-on motion. The insertion direction of the connectors for ports 5, 6 is the same as at least the terminal part of the slide-on motion.

The latch 19, having a locking position and a release position, is biased toward the locking position which prevents removal of the chamber from the CPAP machine. The front face of the latch may be shaped such that during the single slide-on motion employed to fit the water chamber to the CPAP machine, the flange 18 urges the securing latch 19 into the release position and allows the water chamber to be properly fitted. Once the base of the water chamber is properly seated on the heater base and the inlet 5 and outlet 6 are properly engaged with the connection manifold 8, the flange 18 and base of the chamber no longer contact the securing latch 19. This allows the securing latch biasing means to urge the latch into the locking position and prevent the water chamber from being removed as shown in FIG. 2.

Preferably the connection manifold 8 includes a passage which receives pressurised airflow from the blower and directs it into the water chamber 2, and a passage which directs airflow received via the water chamber outlet port 6, to the CPAP patient outlet port 9. The connection passage connecting the manifold inlet port 7, to the manifold patient outlet port 9 is shown in hidden detail 48 in FIG. 1. The connection manifold 8 of the present invention is preferably embodied in a removable component to aid cleaning and/or sterilisation of the gases passageways. In one preferred embodiment the above connection passages are internal to the connection manifold 8 as illustrated in FIGS. 1 and 2.

In use, air from the CPAP machine blower exits through outlet port 4, and enters the chamber 2 through inlet port 5. A chamber heating means 58 vaporises liquid water in the chamber, and air entering the chamber is humidified by the evaporation of water from the water source in the bottom of the chamber before leaving the chamber through the patient outlet port 6. Humidified air from the outlet port 6 is received into the connection manifold of the CPAP machine 8 via the inlet port 7. The connection manifold 8 directs air to the outlet port 9 which is adapted to connect with a flexible conduit connector for delivery to a patient. An advantage obtained from the breathing conduit connection 9 being located on the body of the CPAP machine and not connected to the top of the water chamber directly, is that complete connection or disconnection of the water chamber from the CPAP system (including the breathing conduit) can be achieved with a single slide-on or slide-off motion respectively. This feature simplifies removal of the water chamber for refilling compared with prior art devices.

A further advantage is obtained when additional electrical or pneumatic connections are required for example for heated delivery conduits. The use of heated conduits usually requires electrical wiring connectors between the conduit and humidified air source while an additional pneumatic connection may be used for pressure feedback or measurement. In the present invention the connector may include any additional electrical and/or pneumatic 54 connection for the conduit. The connector is integral to the connection manifold of the CPAP machine 8 and therefore allows the disposable water chamber to remain simple for example lacking electrical transfer connections.

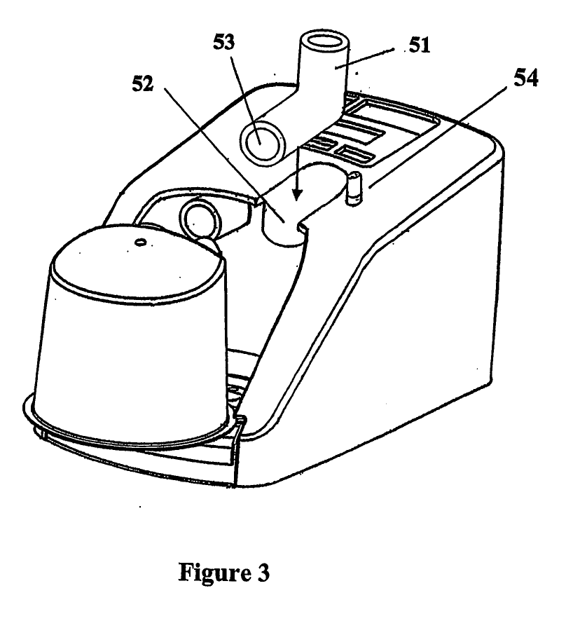

A number of alternative variations of the present invention are envisaged and will now be described. For example, a further embodiment of the present invention is envisaged to deliver humidified gases from the water chamber to a patient via a flexible breathing conduit wherein the humidified gases portion of the manifold is separately removable from the housing. This alternative embodiment is shown in FIG. 3. An elbow tube 51 having an inlet end and an outlet end is provided to receive humidified gases from the water chamber and direct humidified gases into a flexible breathing conduit for delivery to a patient. In this alternative embodiment the CPAP machine housing is provided with a recess 52 for receiving and securing the elbow tube. The recess 52 may include a neck or constriction above the elbow 51, (when elbow 51 is in place) to hold the elbow in place under normal usage, but also allow the elbow to be removed when required. It will be appreciated that other methods of removably securing elbow 51, will readily present themselves to those skilled in the art. For example via various protrusions and interacting slots on one or other of elbow 51, or around recess 8, or both. When secured in position, an inlet 53 of the elbow tube 51 is positioned to make a fluid connection to the outlet 6 of the water chamber in the same slide on motion. In this alternative embodiment the outlet elbow may be part of the termination of the breathing tube instead of an internal part of the connection manifold as previously described. An advantage of this alternative embodiment is that the parts in contact with potential condensation are removable for cleaning and/or sterilisation. This embodiment also retains the advantage of an engagable/disengagable water chamber in a single slide on/off motion. This embodiment may also allow additional electrical or pneumatic connections 54 between the CPAP machine and a conduit connector to be made directly to the housing enabling this alternative to retain the advantages of the previously described embodiments.

An alternative embodiment of the present invention is envisaged wherein a water chamber and heater base are partially or fully enclosed in a housing. The housing includes a connection manifold consisting of at least one gases inlet and at least one gases outlet, connection port being adjacent and aligned, which in use transport gases to and/or from the water chamber. A second housing is provided with complementary inlet and outlet connections for registration with the connection manifold. The second housing is adapted to engage with the first housing making all the necessary gases and electrical or pneumatic connections in the same slide-on motion and preferably includes a securing arrangement to lock the two housings together. The second housing may include an integral air blower, and a patient conduit outlet port in the case of a CPAP embodiment. The first conduit port in use receiving air from a source and the second conduit port delivering humidified air to a patient. The above described embodiment has the advantage that all necessary flexible conduit connections are made on the second housing (incorporating the gases supply). This enables the water chamber and/or enclosing housing to be removed/engaged in the same slide-off/on motion making engagement/disengagement and refilling of the chamber simpler.

In the preferred embodiments of the present invention, tubular protrusions (4, 7) are provided for making a connection between the humidifier apparatus and a water chamber in order to deliver gases to the chamber and receive humidified gases from the chamber. Preferably the tubular protrusions also include a resilient boot in order to provide an improved seal between the water chamber and the protrusions.

A further embodiment of the present invention is envisaged wherein the connections between the apparatus manifold and the water chamber are not provided side by side, but rather are provided one within the other, for example the inlet and outlet may be coaxial. Such a configuration would enjoy the same advantages as the configurations described in more detail in the preferred embodiments of the present invention. It is also envisaged that such connections may also include similarly configured tubes for providing pressure measurements or pressure feedback as well as electrical connections.

While the above preferred embodiments describe male/female type complimentary connectors wherein the water chamber has two female connectors for mating with corresponding male connectors of the apparatus manifold, many variations will present themselves to those skilled in the art without departing from the spirit of the present invention. For example the water chamber may be provided with two male connectors while the apparatus manifold is provided with corresponding female connectors, or the water chamber may be provided with one male and one female connector for connecting to the corresponding male and female connectors of the apparatus manifold. Further it is envisaged that connectors of an androgynous nature may be provided for making connection between the water chamber and the apparatus manifold wherein each connector may include both male type protruding portions and female type recess portions. Such connections may be particularly advantageous when the inlet and outlet is provided one within the other.

With reference to the above embodiments of the present invention, some common features of a water chamber suitable for use with the embodiments described above will now be described in more detail.

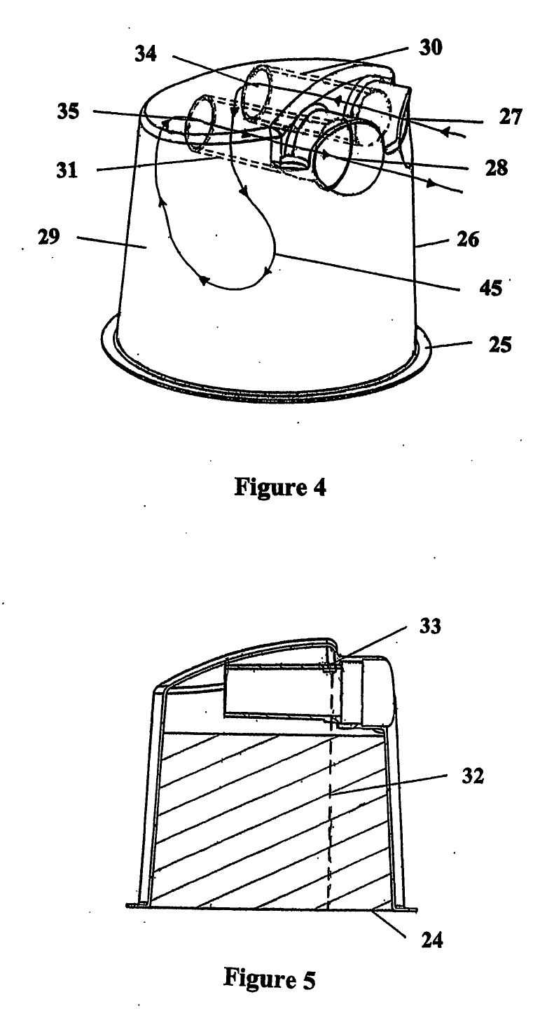

The chamber as shown in FIG. 4 and FIG. 5 is constructed from an open bottomed plastic container enclosed by a heat conductive base 24, and includes a horizontally aligned gases inlet 27 and a parallel gases outlet 28. It is envisaged that other configurations of the present invention are possible where the slide-on direction employed to fit the water chamber is not horizontal but at an angle from the horizontal or vertical. In such cases, the gases inlet 27 and outlet 28, are preferably parallel and aligned with the direction of the intended slide-on motion to allow mating of the chamber inlet/outlet ports and the connection manifold.

The water chamber of the present invention preferably includes at least one flow tube, being an inlet extension tube 30, and/or an outlet extension tube 31, extending inwardly into the chamber interior from the periphery of the chamber wall and preferably having a generally tapering body. The inlet extension tube 30 and the outlet extension tube 31 are preferably moulded from the same clear thermoplastic material as the chamber shell 26. The inclusion of inlet/outlet extension tubes has been found to significantly reduce noise produced by the airflow around the chamber. However at high flow rates, it is possible for water droplets or splashes to become entrained in the air flow and be carried out the chamber outlet 28. This is especially possible when the water chamber contains a large amount of liquid and the water surface is closer to the chamber outlet. This situation has the potential to become more problematic if the outlet port of the CPAP machine is disconnected from the patient delivery conduit, lowering the circuit resistance and resulting in significantly higher flow rates. Further, without the delivery conduit connected, any liquid entrained in the gases flow may be ejected directly from the chamber. This difficulty may be alleviated somewhat in chambers incorporating various extension tube configurations.

Preferably at least one extension tube has an air bleed aperture 33 to aid filling of the chamber with the chamber tipped up. The air bleed is preferably located in the top surface of the extension tube and preferably toward the end of the extension tube which is connected to the chamber wall. Referring to FIG. 5, preferably the air bleed aperture 33 is positioned such that when the tank is tipped up for filling, the air bleed valve height corresponds with the preferred fill height 32 for the water chamber. This feature aids in preventing overfilling of the water chamber.

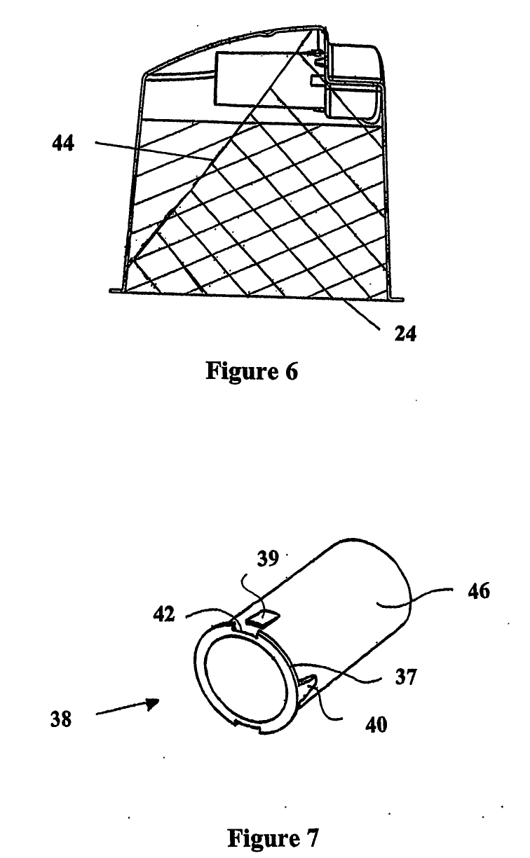

Additionally, with reference to FIG. 6, the extension tubes 30 and 31 may act as a weir against water flow back through the gases inlet and gases outlet, upon tilting of the chamber as shown by water level line 44. This reduces water back-flow through the inlet port 27 occurring upon tilting of the chamber. If present, preferably the air bleed aperture 33 is present only on the outlet extension tube 31 and not present in the inlet extension tube 30. Alternatively the air bleed aperture may be included on both.

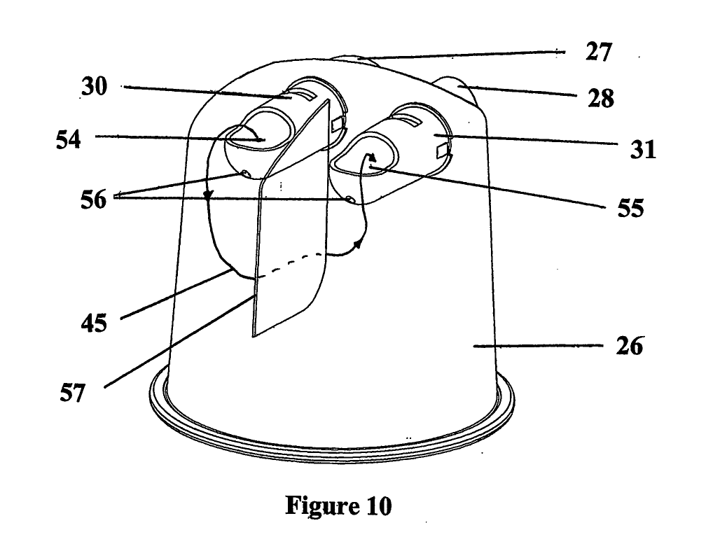

With reference to FIG. 10, the present invention may further include a downwardly extending central baffle or rib 57 located between the inlet and outlet extension tubes to ensure against gases short circuiting the chamber by flowing directly from the exit of the inlet extension tube, to the entry of the outlet extension tube. With the baffle present, the gases are forced to follow a more tortuous path ensuring adequate humidification during their journey through the chamber but without increasing the pressure losses in the chamber to an unacceptable level. The baffle preferably extends downwards from the roof of the chamber, and inwards from the portion of the chamber wall opposite the inlet/outlet port. Preferably the size of the baffle is such that it not only ensures that the gases flow follows a torturous path through the chamber, but also provides an additional barrier to splashes entering the inlet 55 of the outlet extension tube 31. As the risk of splashes entering the extension tubes is highest when the water level is highest, the baffle may extend downwards such that it terminates below the water line when the chamber is full.

With reference to FIG. 4, in use air is received into the chamber via inlet port 27 and travels down the inlet extension tube 30. On exiting the inlet extension tube 30 air enters the chamber where it is humidified by the evaporation of water from the water supply. Humidified air flows from the chamber through the outlet extension tube 31 and exits through outlet port 28 as illustrated by arrow 45. With reference to FIG. 10, an alternative configuration of the extension tubes wherein the distal end of the extension tube furthest from the gases inlet 27 and gases outlet 28 respectively are directed away from the axis of the extension tube. The extension tubes are shaped to minimise the internal pressure losses of the gases flowing through the chamber in order to improve the efficiency of the chamber. In use, air is received into the chamber via inlet port 27 and travels down the inlet extension tube 30. On exiting the upwardly facing outlet 54 of the inlet extension tube 30, the gases flow is directed away from the surface of the water in the chamber, minising the potential for splashing or water entrainment to occur. As the gases flow enters the chamber it is deflected off the roof of the chamber and is humidified by the evaporation of water from the water supply. Humidified air flows from the chamber through the upwardly facing inlet 55 of the outlet extension tube 31 and exits through outlet port 28. The upwardly oriented inlet 55 of the outlet extension tube 31 eliminates the direct path that splashes might have from the surface of the water into the outlet port 28. A drain hole 56 is provided in the bottom of the extension tubes to enable water to drain back into the chamber after filling, or built up condensation or splashes to drain during use. Preferably the shape and orientation of the extension tube and the position of the drain hole are such that the drain hole is at a low point and fluid flows toward the drain hole and back into the chamber.

Alternatively, it is envisaged that the direction in which the outlet of the inlet extension tube and/or the inlet of the outlet extension tube, faces could be varied in order to achieve differing results. For example, the openings at the distal ends of the extension tubes may be rotated about the axis of the extension tube, to face in any direction. Further, the direction in which the openings of the inlet and outlet flow tubes face may not be the same. Such arrangements (for example facing mutually away from each other) maybe particularly suited for reducing the potential for splashes, and reducing the potential for splashes to enter the opening of the extension tubes when the baffle is present. Although the preceding description gives details of preferred embodiments having parallel and adjacent circular inlet/outlet ports, it is envisaged that other configurations are possible without departing from the spirit of the invention. For example the inlet/outlet ports of the chamber and connection manifold may have a non-circular cross section and not be symmetrical. Further it is possible that the position of the inlet port with respect to the outlet may take one of many alternative configurations. For example the ports and there corresponding connections may also be co-axial or off-set, one inside the other.

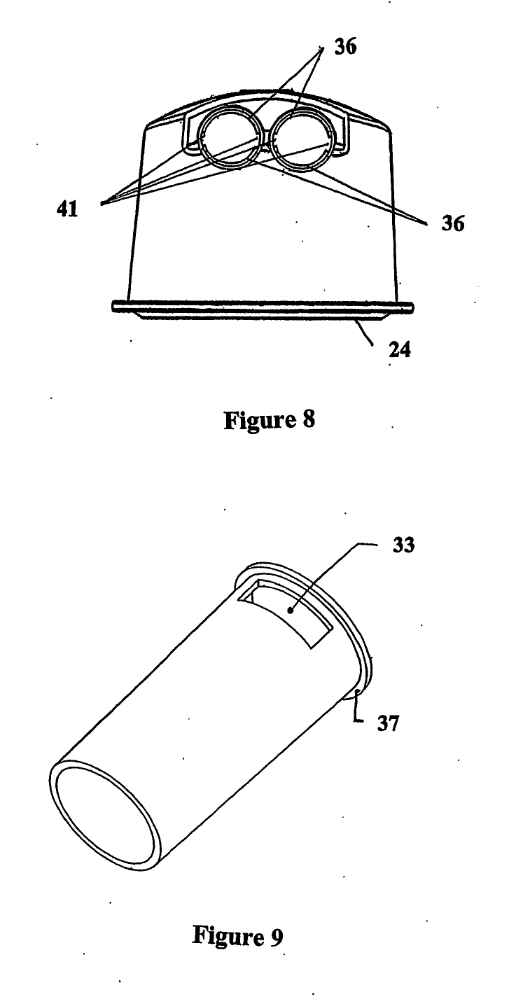

Referring to FIGS. 7-9, for ease of assembly the inlet and outlet extension tubes are preferably provided as a snap fit to their respective water chamber inlet or outlet, so that they can be pushed into the chamber through the inlet or outlet and, upon application of sufficient force, snap into a substantially watertight and secure condition.

To this end the inlet 27 and outlet 28 ports of the water chamber may be provided with an inwardly perpendicularly extending annular flange 36 at the inner end thereof and the inlet/outlet extension tubes 38 may include similar perpendicularly outwardly extending flanges 37 from one end of the generally tapering tubular body 46. The flanges act together as sealing flanges in the fitted and assembled condition. To retain the extension tubes in the assembled condition, against both translational and rotational movement several securing mechanisms may be provided. In each case the securing mechanisms may be provided on either of the inlet/outlet (of the chamber) or the inlet/outlet extension tube. However it is preferred that they be on the extension tubes, as both components are intended for injection moulding and injection moulding of certain protrusions on the inner surface of the chamber inlet/outlet would be considerably more difficult than on the outer surface of the extension tubes. To secure the tubes against translational movement, and in a sealing condition between the sealing flanges, a plurality of retaining clip protrusions 39 may be provided spaced around the circumference of the tubular body of the extension tubes which co-operate with the inlet/outlet flange 36. Particularly for ease of manufacture, and ensuring a simple two part injection mould, a notch 42 is allowed in the flange 37 of the extension tubes 38 adjacent the protrusion 39.

To retain the extension tubes against rotational movement when snap fitted into location, one or more locating protrusions 40 may be provided circumferentially distributed on the outer surface of the tubular body adjacent and contiguous with the outwardly and perpendicularly extending flange 37. The locating protrusions 40 are preferably generally tapered in both the circumferential and axial direction. Complementary notches 41 are provided in the inwardly extending flanges 36 of the chamber inlet and outlet. In fitting the extension tubes 38 the protrusions 40 are aligned with the notches 41, and upon full insertion of the tubes, the protrusions 40 enter into a tight frictional fit with the notches 41 ensuring substantial if not complete sealing. It will be appreciated that the mechanism employed to ensure proper location and sealing of the extension tubes into the water chamber may take many forms. Many alternatives will suggest themselves to persons skilled in the art such as glued joints, various forms of plastic welding and various configurations of clipping means and protrusions. The above description is of one particular preferred embodiment and is not meant to be in any way limiting.

It will be readily appreciated that the construction of the water chamber as described is simple to manufacture and each of the plastic components is itself capable of simple injection moulding. Consequently a water chamber according to the present invention is, while providing significant advantages, not significantly more expensive than existing chambers.

1. An apparatus for use in humidified gases delivery treatment comprising:
a housing,
a pressurised gases supply within said housing,
a pressurised gases outlet in said housing in fluid connection with said pressurised gases supply and adapted to make fluid connection with an inlet of a heatable water chamber in order to provide gases flow to said chamber,
a humidified gases return in said housing, adapted to make fluid connection with an outlet of said chamber in order to receive humidified gases from said chamber,
a chamber heating means connected to said housing and, said housing includes a humidifier engagement locating said chamber adjacent said chamber heating means, said chamber heating means adapted to vaporise liquid water in said water chamber in order to provide water vapour to said gases flow passing through said water chamber,
a patient outlet in said housing, in fluid connection with said humidified gases return in order to receive humidified gases from said humidified gases return and provide humidified gases to said patient outlet, said patient outlet being in fluid connection with or adapted to make fluid connection with a breathing conduit for delivery of humidified gases to a patient, and wherein
said humidification chamber has a base, and said chamber is engagable with said humidifier engagement via a single motion, and said single motion of engagement urges the base of said humidification chamber adjacent and in contact with said chamber heating means and makes a first fluid connection between said pressurised gases outlet and said humidifier inlet, and makes a second fluid connection between said humidified gases return and said humidifier outlet, with said first and second fluid connections being made in the direction of said single motion.

2. An apparatus for use in humidified gases delivery treatment as claimed in claim 1, wherein said pressurised gases outlet and said inlet of a said humidifier have between them first complementary male and female connectors, having a preferred insertion direction for completing a fluid connection by engagement of the male and female connectors,
said humidified gases return and said outlet of said humidifier have between them second complementary male and female connectors, having a preferred insertion direction for completing a fluid connection by engagement of the male and female connectors, said preferred insertion direction of said first connectors being the same as said preferred insertion direction of said second connectors, and being the same as at least the direction of a terminal part of said single motion.

3. An apparatus for use in humidified gases delivery treatment as claimed in claim 2 wherein said inlet of said humidifier and said outlet of said humidifier are each a female port,
and said pressurised gases outlet and said humidified gases return are each a resilient tubular projection fitting within respective female ports with said chamber engaged.

4. An apparatus for use in humidified gases delivery treatment as claimed in claim 3 wherein said protruding tubes of said pressurised gases outlet and humidified gases return have substantially parallel axis of extension, said chamber heating means includes a substantially planar heating plate, and said axis of extension of said tubes are at least substantially parallel with the plane of said heating plate.

5. An apparatus for use in humidified gases delivery treatment as claimed in any one of claims 1-4 wherein said patient outlet includes a connector for receiving a breathing hose and at least one auxiliary electrical connection plug or socket or pneumatic connection plug or prot, for a simultaneous connection when connecting a breathing circuit having complementary electrical or pneumatic connectors.

6. A humidifier chamber for use with a gases humidification apparatus comprising:
a container, with a surrounding wall and top, and an open bottom,
a heat conductive base enclosing said open bottom of said container,
a gases inlet to said container,
a gases outlet to said container,
a first elongate flow tube extending into said humidifier container from the inner periphery of said gases inlet, with an opening at a distal end of said flow tube being spaced from said wall of said chamber,
a second elongate flow tube extending into said humidifier container from the inner periphery of said gases outlet, with an opening at a distal and of said flow tube being spaced from said wall of said chamber,
said first and second flow tubes being substantially parallel to each other, and substantially parallel to said base of said chamber, and
said gases inlet and said gases outlet facing the same direction, a preferred insertion direction, and
said preferred insertion direction is substantially parallel to the said base of said chamber, such that
said humidifier chamber may make operable engagement with a heater base in a single motion,
and fluid connection with said gases outlet and said gases inlet, being also made in said single motion.

7. A humidifier chamber as claimed in claim 6, wherein said opening of said first flow tube faces a direction transverse to an axis of said first flow tube, and said opening of said second flow tube faces a direction transverse to an axis of said second flow tube.

8. A humidifier chamber as claimed in claim 7, wherein said transverse direction is not downwards.

9. A humidifier chamber as claimed in claim 7, wherein said transverse direct is upwards.

10. A humidifier chamber as claimed in claim 6, wherein said chamber further includes a baffle between said first flow tube and said second flow tube.

11. A humidifier chamber as claimed in claim 10, wherein said baffle extends from the roof of said chamber and terminates below the surface of water in said chamber when said chamber is filled to a maximum intended water level for use.

12. A humidifier chamber as claimed in claim 6, wherein said second tube includes an air bleed orifice, said air bleed orifice being located in the top of said second elongate flow tube, and located toward the end of the elongate flow tube adjacent said gases outlet.

13. A humidifier chamber as claimed in any one of claims 6 to 12, wherein said gases inlet and said gases outlet of said humidifier chamber are each a female port, and
said humidifier chamber is generally cylindrical, and said female ports open out to the cylindrical surface adjacent the top of the cylindrical wall.

14. (canceled)

15. (canceled)
